(19) United States
(12) Patent Application Publication
(10) Pub. No.: US 2014/0154187 A1
(43) Pub. Date: Jun. 5, 2014

(54) Liquid Tooth Paste

(71) Applicant: Bonnie Carol Douglas, Parma, OH (US)

(72) Inventor: Bonnie Carol Douglas, Parma, OH (US)

(21) Appl. No.: 14/174,708

(22) Filed: Feb. 6, 2014

Related U.S. Application Data
(63) Continuation-in-part of application No. 13/317,680, filed on Jul. 6, 2012.

(51) Int. Cl. A61K 8/73; A61K 8/34; A61Q 11/00

(52) U.S. Cl. CPC A61K 8/731; A61Q 11/00; A61K 8/73; A61K 8/345
USPC 424/49

(57) ABSTRACT

My tooth cream will not stick, this is what makes it different from other tooth pastes. It can be dispensed in any type container, but it is different in that this toothpaste contains NO thickeners, no carrageenan, no cellulose gum, no glycerin and no SLS (sodium lanyl sulfates). My tooth crème can contain colorings, flavorings, preservatives, xylitol, sweetnings, fluoride . . . infinity.

### CROSS-REFERENCE TO RELATED APPLICATION

This application is a continuation-in-part application of co-pending U.S. patent application Ser. No. 13/317,680, filed Jul. 6, 2012, the content of which is incorporated by reference in its entirety.

### SUMMARY OF THE INVENTION

One aspect of the invention is a liquid toothpaste formula which has a reduction or near elimination from the standard toothpaste formula of certain additives that make toothpaste stiff, cement-like and sticky. The additives include, but are not limited to, carrageenan, cellulose gum (carboxymethyl cellulose (CMC)), and glycerin. This formula reduces one or more of such additives in an amount sufficient to reduce or eliminate thickness of conventional toothpaste rendering the tooth “paste” liquid.

### DETAILED DESCRIPTION OF THE INVENTION

Before the present invention is disclosed and described, it is to be understood that the aspects described below are not limited to specific compositions, methods or preparing such compositions, or uses thereof as such may, of course, vary. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise. Thus, for example, reference to “a binder” includes mixtures of two or more binders, and the like.

“Optional” or “optionally” means that the subsequently described event or circumstance can or cannot occur, and that the description includes instances where the event or circumstance occurs and instances where it does not. For example, the phrase “optional cleaning step” means that the cleaning step may or may not be performed.

The term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “about” when used before a numerical value indicates that the value may vary within a reasonable range, such as ±5%, ±1%, ±0.5%, and ±0.2%.

Unless otherwise indicated, the percentage of an ingredient in the toothpaste refers to weight percentage of the ingredient relevant to the total weight of the liquid toothpaste.

In one aspect, the toothpaste of this invention is a toothpaste that has a reduction or near elimination of certain additives that make standard toothpaste stiff, cement-like and sticky. In one aspect, the toothpaste formula reduces or eliminates carrageenan, cellulose gum, glycerin, in an amount and various degrees enough of a reduction (such as at least 50% reduction) in these elements to render the tooth “paste” liquid.

The toothpaste will have zero or very small amounts of glycerin, carrageen and cellulose gum in order to make it in a liquid state—non-sticky, non-cementable to be dispensed in a sanitary pump similar but not limited to, a liquid soap dispenser container.

The formula of this invention is in a liquid form, with/without abrasives, with/without sodium lauryl sulfate (SLS), can include sudser, colorings, flavorings, preservatives, sorbital, water, xylitol, sweeteners, fluoride, etc.

In another aspect, provided is an improved toothpaste, wherein the improvement comprises a reduction of carrageenan, cellulose gum and/or glycerin of a standard toothpaste, such as to an amount described herein, thereby rendering the toothpaste suitable for application via a liquid dispenser and further rendering the toothpaste non-sticky to hair, clothing, sinks, floors, towels, rugs, or walls.

In some embodiments, the toothpaste has at least 50% reduction of a binder than regular toothpaste. In some embodiments, the toothpaste has at least 60%, 70%, 80%, 90% or 95% reduction of a binder than regular toothpaste. In some embodiments, the binder is present in the liquid toothpaste formula of the invention in an amount that is less than 50% of the amount in a regular toothpaste. In some embodiments, the binder is present in the formula in an amount that is less than 45%, less than 40%, less than 35%, less than 30%, less than 25%, less than 20%, less than 15%, less than 10%, less than 5%, or less than 1% of the amount in a regular toothpaste. In some embodiments, the binder is present in the toothpaste in an amount that is about 45%, about 40%, about 35%, about 30%, about 25%, about 20%, about 15%, about 10%, about 5%, or about 1% of the amount in a regular toothpaste, or any range between any two of these values (including endpoints). In some embodiments, the formula does not have a binder. Examples of binders include but are not limited to carrageenan, cellulose gum and glycerin.

In some embodiments, the abrasive is present in the liquid toothpaste formula of the invention in an amount that is less than about 50% of the amount in a regular toothpaste. In some embodiments, the abrasive is present in an amount that is from about 50% to substantially the same as the amount in a regular toothpaste, such as 50%, 60%, 70%, 80%, 90%, 95%, 100%, 105%, or 110% of the amount in a regular toothpaste, or any range between any two of the values, end points inclusive. In some embodiments, the toothpaste does not comprise fluoride. In some embodiments, the toothpaste comprises from about 50% to substantially the same levels of fluoride as regular toothpaste, such as 50%, 60%, 70%, 80%, 90%, 95%, 100%, 105%, or 110% of the amount in a regular toothpaste, or any range between any two of the values, end points inclusive.

In some embodiments, the toothpaste has at least 50% reduction of a sudser than regular toothpaste. In some embodiments, the toothpaste has at least 60%, 70%, 80%, 90% or 95% reduction of a sudser than regular toothpaste. In some embodiments, the liquid toothpaste comprises a sudser in an amount that is less than 50% of the amount of sudser in regular toothpaste. In some embodiments, the liquid toothpaste comprises a sudser that is less than about 50% of the amount of suder in regular toothpaste. In some embodiments, the sudser is present in the formula in an amount that is less than 45%, less than 40%, less than 35%, less than 30%, less than 25%, less than 20%, less than 15%, less than 10%, less than 5%, or less than 1% of the amount in a regular toothpaste. In some embodiments, the sudser is present in the toothpaste in an amount that is about 45%, about 40%, about 35%, about 30%, about 25%, about 20%, about 15%, about 10%, about 5%, or about 1% of the amount in a regular toothpaste, or any range between any two of these values (including endpoints). In some embodiments, the liquid toothpaste comprises an abrasive in an amount that is the same as in regular toothpaste, such as 50%, 60%, 70%, 80%, 90%, 95%, 100%, 105%, or 110% of the amount in a regular toothpaste, or any range between any two of the values, end points inclusive. In some embodiments, the liquid toothpaste comprises fluoride in an amount of from zero to about the same as in regular toothpaste, such as 50% of the levels of regular toothpaste. In some embodiments, the liquid toothpaste comprises fluoride in an amount of about 50%, 60%, 70%, 80%, 90%, 95%, 100%, 105%, or 110% of the amount in a regular toothpaste, or any range between any two of the values, end points inclusive.

In some embodiments, the liquid toothpaste is suitable for application to a toothbrush, and is not stiff and non-sticky to, and will not stick to hair, clothing, sinks, floors, towels, rugs, or walls.

In some embodiments, the liquid toothpaste has a viscosity of no more than about 2000 centipoise, such 1 centipoise, 5 centipoise, 10 centipoise, 20 centipoise, 25 centipoise, 50 centipoise, 70 centipoise, 100 centipoise, 120 centipoise, 150 centipoise, 170 centipoise, 200 centipoise, 500 centipoise, 700 centipoise, 1000 centipoise, 1200 centipoise, 1500 centipoise, 1700 centipoise, or 2000 centipoise, or any range between any two of the values, including end points, such as from 1 centipoise to 2000 centipoise, from 5 centipoise to 1000 centipoise, from 10 centipoise to 200 centipoise, from 1 centipoise to 1000 centipoise, from 1 centipoise to 500 centipoise, from 1 centipoise to 100 centipoise, from 1 centipoise to 50 centipoise, from 1 centipoise to 25 centipoise, at 20° C.

In some embodiments, the liquid toothpaste comprises from 0% to 0.05% carrageenan, or from about 0% to about 0.04%, 0% to about 0.03%, 0% to about 0.02%, or 0% to about 0.01% carrageenan. In some embodiments, the formula comprises 0%, 0.001%, 0.002%, 0.003%, 0.004%, 0.005%, 0.006%, 0.007%, 0.008%, 0.009%, 0.01%, 0.015%, 0.02%, 0.025%, 0.03%, 0.035%, 0.04%, 0.045% or 0.05% of carrageenan, or any range between two of the values (end points inclusive). In some embodiments, the formula comprises from about 0% to about 0.1% cellulose gum. In some embodiments, the formula comprises 0%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.11%, or 0.12% of cellulose gum, or any range between two of the values (end points inclusive). In some embodiments, the formula comprises from about 0% to about 0.01% cellulose gum, from about 0% to about 0.02% cellulose gum, from about 0% to about 0.03% cellulose gum, from about 0% to about 0.04% cellulose gum. In some embodiments, the formula comprises about 0% to about 4% of glycerin. In some embodiments, the formula comprises 0%, 0.001%, 0.005%, 0.01%, 0.02%, 0.05%, 0.07%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or 4% of glycerin, or any range between two of the values (end points inclusive). In some embodiments, the formula comprises from about 0% to about 3% glycerin, from about 0% to about 2% glycerin, from about 0% to about 1% cellulose gum, from about 0% to about 0.05% glycerin. In some embodiments, the formula comprises about 0% to about 0.2% xanthan gum. In some embodiments, the formula comprises 0%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.092%, 0.1%, 0.12%, 0.13%, 0.14%, 0.15%, 0.16%, 0.17%, 0.18%, 0.19% or 0.2% of xanthan gum, or any range between two of the values (end points inclusive). In some embodiments, the formula comprises about 0% to about 0.1%, about 0.001% to about 0.1%, about 0.01% to about 0.1%, about 0.001% to about 0.01% xanthan gum.

In some embodiments, the formula comprises about 5% to about 40% of an abrasive. In some embodiments, the formula comprises 5%, 10%, 15%, 20%, 25%, 30%, 35% or 40% of an abrasive, or any range between two of the values (end points inclusive). Abrasives include but are not limited to aluminum silicate, aluminum hydroxide, bentonite, calcium carbonate, calcium hydrogen phosphate, calcium phosphate, hydroxyapatite, potassium metaphosphate, silicas, sodium metaphosphate and zeolites. In some embodiments, the abrasive is hydrated silica.

In some embodiments, the formula further comprises a fluoride compound, such as ammonium fluorosilicate, potassium fluoride, sodium fluoride, sodium fluorosilicate, sodium monofluorophosphate, and stannous fluoride. In some embodiments, the concentration of the fluoride ion in the formula is from 0 ppm to 2500 ppm, such as 10 ppm, 100 ppm, 200 ppm, 500 ppm, 1000 ppm, 1500 ppm, 2000 ppm, 2500 ppm, or any range between any two values (end points inclusive).

In some embodiments, the formula comprises an antitartar agent, antibacterial such as triclosan or zinc chloride, or other active ingredients that may be present in regular toothpaste.

In some embodiments, the formula comprises about 0% to about 3% surfactant. In some embodiments, the formula comprises about 0%, 0.5%, 1%, 1.5%, 2%, 2.5%, or 3% of a surfactant, or any range between two of the values (end points inclusive). In some embodiments, the surfactant is present in an amount of from 0.05 to 1.5%, 0.1 to 1.0% or 0.1 to 0.25% based on the weight of the liquid toothpaste. In some embodiments, the surfactant is present in an amount of no more than about 0.2%. Examples of surfactant includes cationic, anionic, nonionic, amphoteric or zwitterionic surfactants, such as C₈-C₁₆ fatty acid glutamate salt, a C₈-C₁₆ alkyl glucoside, such as a C₁₀-C₁₄ alkyl glucoside, in particular lauryl glucoside, alkyl sulfate salt, in particular sodium lauryl sulfate (SLS).

In some embodiments, the formula may comprise a coloring agent, a flavoring agent and/or sweetener in an amount as in regular toothpaste, for example, at about 0.1% to about 2% of the total composition. Flavoring agents include but are not limited to peppermint, spearmint, menthol, cinnamon, lemon oil, carvone and wintergreen. Sweeteners include but are not limited to natural or synthetic sweeteners, such as lactose, fructose, maltose, saccharin, sucrose and L-aspartyl-L-phenyl alanine methyl ester. Coloring agents include but are limited to titanium dioxide that makes the liquid toothpaste white, artificial dyes and pigments that make the liquid toothpaste red, green, or blue, for example.

In some embodiments, the formula may comprise a preservative such as ethylparaben, methylparaben and sodium benzoate, in an amount as in regular toothpaste.

The rest of the formula comprises water and/or other solvents, such as sorbitol, xylitol. The formula may further comprise a pH adjusting agent or buffer, such as citric acid and salts thereof or phosphoric acid and salts thereof.

In some embodiments, the formula comprises glycerin, sorbitol and xylitol or a mixture thereof. In some embodiments, the formula comprises from about 0 to about 20% sorbitol. In some embodiments, the formula comprises 0%, 1%, 2%, 5%, 7%, 10%, 12%, 15%, 17%, 20%, 25%, 30%, 35% or 40% sorbitol, or any range between two of the values (end points inclusive). In some embodiments, the formula comprises from about 0 to about 10% xylitol. In some embodiments, the formula comprises 0%, 0.5%, 1%, 2%, 3%, 4%, 5%, 6%, 7%, 8%, 9%, or 10% xylitol, or any range between two of the values (end points inclusive). If the composition comprises a mixture of two or three of sorbitol, glycerin and xylitol, the amount of each may be about 30% to about 70% of their respective amount when only one of sorbitol, glycerin and xylitol is present.

Due to the softer, more liquid form of this toothpaste, this will not stick to sinks, floors, clothing, hair, etc. There will be no messy, daily cleanup from the sticky, cement like quality of regular toothpaste.

In some embodiments, the toothpaste is dispensable from a liquid pump, such as a liquid pump.

In another aspect, provided is a toothbrush having the toothpaste described herein on or in the brush thereof.

In still another aspect, provided is a method of dispensing the toothpaste described herein, wherein the toothpaste is dispensed from a container holding the toothpaste in a liquid dispenser.

The liquid toothpaste of the invention can be prepared by mixing the ingredients described above. The prepared liquid toothpaste can be put into a liquid dispenser where the toothpaste can be dispensed to a toothbrush when being used.

What is claimed is:

1. A toothpaste that has at least 50% reduction of a binder than regular toothpaste.

2. The toothpaste of claim 1, having at least 75% reduction of a binder than regular toothpaste.

3. The toothpaste of claim 1, having at least 90% reduction of a binder than regular toothpaste.

4. The toothpaste of claim 1, having at least 95% reduction of a binder than regular toothpaste.

5. The toothpaste of claim 1, wherein the binder is carrageenan, cellulose gum or glycerin.

6. The toothpaste of claim 1, comprising from 0% to about 0.05% carrageenan.

7. The toothpaste of claim 1, comprising from 0% to about 0.1% cellulose gum.

8. The toothpaste of claim 1, comprising from 0% to about 4% of glycerin.

9. The toothpaste of claim 1, comprising an abrasive in an amount that is from about 50% to substantially the same as the amount in a regular toothpaste.

10. The toothpaste of claim 1 which is dispensable from a liquid pump.

11. A toothbrush having the toothpaste of claim 1 on or in the brush thereof.

12. A toothpaste that has at least 50% reduction a sudser than regular toothpaste.

13. The toothpaste of claim 12, having at least 75% reduction of a sudser than regular toothpaste.

14. The toothpaste of claim 12, having at least 90% reduction of a sudser than regular toothpaste.

15. The toothpaste of claim 12, having at least 95% reduction of a sudser than regular toothpaste.

16. The toothpaste of claim 12, comprising an abrasive in an amount that is from about 50% to substantially the same as the amount in a regular toothpaste.

17. The toothpaste of claim 12, which is dispensable from a liquid pump.

18. A toothbrush having the toothpaste of claim 12, on or in the brush thereof.
